(19) United States
(12) Patent Application Publication
(10) Pub. No.: US 2026/0083765 A1
(43) Pub. Date: Mar. 26, 2026

(54) COMPOSITIONS FOR SUPPORTING, OPTIMIZING AND/OR IMPROVING NAD+ CONCENTRATIONS AND RELATED METHODS

(71) Applicant: Qualia Life Sciences LLC, Carlsbad, CA (US)

(72) Inventor: Gregory Kelly, Oceanside, CA (US)

(21) Appl. No.: 18/891,204

(22) Filed: Sep. 20, 2024

(51) Int. Cl. A61K 31/706; A61K 31/05; A61K 31/197; A61K 31/4188; A61K 31/4415; A61K 31/455; A61K 31/51; A61K 31/522; A61K 31/525; A61K 31/714; A61K 33/06; A61K 36/064; A61P 43/00

(52) U.S. Cl. CPC A61K 31/706; A61K 31/05; A61K 31/197; A61K 31/4188; A61K 31/4415; A61K 31/455; A61K 31/51; A61K 31/522; A61K 31/525; A61K 31/714; A61K 33/06; A61K 36/064; A61P 43/00

(57) ABSTRACT

A composition for supporting nicotinamide adenine dinucleotide (NAD+) concentrations in humans is provided. The composition includes a synergistically effective amount of: nicotinamide riboside, nicotinamide and nicotinic acid; at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and at least one of magnesium, trans-resveratrol and caffeine. Related methods are also provided.

### FIELD OF THE INVENTION

This disclosure is related to compositions that support, optimize and/or improve NAD+ concentrations in animals, mammals and/or humans and related methods.

### BACKGROUND OF THE INVENTION

A cell is fundamentally a unit of life. We are built out of cells. And our health and vulnerabilities stem from the health or vulnerability of our cells. These cells rely on a coenzyme called nicotinamide adenine dinucleotide (NAD+) to carry out several hundred metabolic functions. Accordingly, NAD+ is a major node, or hub molecule, for cellular function and repair, mitochondrial performance, and healthy aging. In addition to a host of other support roles in the body, NAD+ is integrally involved in energy creation (e.g., ATP production), cellular protection and detoxification, maintaining healthy DNA, and fueling cellular stress-response pathways. In fact, NAD+ fuels important stress and nutrient-sensing pathways that cells rely on to adapt to their environment. NAD+ is used in all of our cells, and it's used by the trillions of microbes in our gut microbiome. Accordingly, NAD+ is indispensable for life.

For these and many other reasons, the NAD+ molecule is central to cellular and mitochondrial health such that attaining and maintaining cellular health requires sufficient NAD+. And stress of any type taxes the cell's NAD+ pool. In response, the cell will attempt to upregulate NAD+ production to adapt to the stress. However, scientific research strongly suggests our cells do not always have the resources needed to meet the increased NAD+ demand.

Some people have suboptimal NAD+ levels, even at younger ages (e.g., 20 years old). However, aging exacerbates suboptimal NAD+ levels. Many of us will experience a sharp decline in NAD+ levels by around thirty years of age. The vast majority of people in their 40's are relatively low in NAD+ compared to a healthy 20 year-old. And if you're 50 years old or older, you likely have about half or less of the NAD+ levels you had when you were younger in at least some of our tissues.

In fact, the decline in NAD+ with age is so consistent that it has been proposed as a biomarker of aging. There are twelve proposed Hallmarks of Aging-characteristics of aging shared by all organisms. Several of the proposed hallmarks, such as mitochondrial dysfunction, genomic instability, deregulated nutrient-sensing, and stem cell exhaustion, are known to be directly impacted by NAD+ availability. It has even been proposed that declines in NAD+ may contribute to all twelve proposed Hallmarks of Aging. Declines in NAD+ also appear to be a leading indicator of declines in mental and physical performance. Accordingly, having less NAD+ to draw on may make our cells more susceptible to at least some, and potentially all, of the Hallmarks of Aging.

Accordingly, there is a need for supporting and/or boosting NAD+ concentrations in animals, mammals and humans to maintain healthier states and reduce vulnerability to stressors of all types.

### SUMMARY OF THE INVENTION

In some embodiments, a composition for supporting nicotinamide adenine dinucleotide (NAD+) concentrations in humans is provided. The composition includes a synergistically effective amount of: nicotinamide riboside, nicotinamide and nicotinic acid; at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and at least one of magnesium, trans-resveratrol and caffeine.

In some embodiments, the composition includes: less than 30 mg of nicotinic acid; between 100-500 mg of niacinamide; between 132-880 mg of nicotinamide riboside or between 150-1000 mg of nicotinamide riboside chloride; between 0.16-6 mg of thiamin (vitamin B1); between 0.2-6 mg of riboflavin (vitamin B2); between 0.65-10 mg of pantothenic acid (vitamin B5); between 0.24-8 mg of pyridoxine (vitamin B6); between 0.15-30 mcg of biotin (vitamin B7); between 50-800 mcg of folate (vitamin B9); between 0.5-10 mcg of cobalamin (vitamin B12); less than 50 mg of caffeine; between 8-200 mg of trans-resveratrol; and between 25-167 mg of magnesium.

In some embodiments, the composition includes: between 8-29 mg of nicotinic acid; approximately 234 mg of niacinamide; and approximately 264 mg of nicotinamide riboside or approximately 300 mg of nicotinamide riboside chloride. In some embodiments, the composition includes: approximately 25 mg of nicotinic acid; approximately 234 mg of niacinamide; and approximately 264 mg of nicotinamide riboside or approximately 300 mg of nicotinamide riboside chloride. In some embodiments, the composition includes: approximately 16 mg of nicotinic acid; approximately 234 mg of niacinamide; and approximately 264 mg of nicotinamide riboside or approximately 300 mg of nicotinamide riboside chloride. In some embodiments, the composition includes: approximately 0.32 mg of thiamin (vitamin B1); approximately 0.4 mg of riboflavin (vitamin B2); approximately 1.375 mg of pantothenic acid (vitamin B5); approximately 0.49 mg of pyridoxine (vitamin B6); approximately 37.5 mcg of biotin (vitamin B7); approximately 166 mcg DFE of folate (vitamin B9); and approximately 1.5 mcg of cobalamin (vitamin B12). In some embodiments, the composition includes postbiotic yeast comprising the at least one of riboflavin (vitamin B2), pantothenic acid (vitamin B5), pyridoxine (vitamin B6), biotin (vitamin B7), folate (vitamin B9) and cobalamin (vitamin B12). In some embodiments, the postbiotic yeast comprise Saccharomyces cerevisiae. In some embodiments, the composition includes: approximately 28 mg of caffeine; approximately 50 mg of trans-resveratrol; and approximately 50 mg of magnesium. In some embodiments, the composition includes less than 40 mg of caffeine. In some embodiments, the composition includes between 10-39 mg of caffeine. In some embodiments, the composition includes whole coffee fruit extract, which comprises the caffeine. In some embodiments, the composition is further disposed within at least one pharmaceutically acceptable vehicle. In some embodiments, the composition is a powder.

In some embodiments, a method of supporting nicotinamide adenine dinucleotide (NAD+) concentrations in humans is provided. The method includes orally administering a composition comprising a synergistically effective amount of: nicotinamide riboside, nicotinamide and nicotinic acid; at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and at least one of magnesium, trans-resveratrol and caffeine. In some embodiments, the method further includes measuring NAD+ concentration in the body.

In some embodiments, a method of manufacturing a composition for supporting nicotinamide adenine dinucleotide (NAD+) concentrations in humans is provided. The method includes combining a synergistically effective amount of: nicotinamide riboside, nicotinamide and nicotinic acid; at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and at least one of magnesium, trans-resveratrol and caffeine. In some embodiments, the method further includes disposing the composition within at least one pharmaceutically acceptable vehicle.

### BRIEF DESCRIPTION OF THE FIGURES

FIG. 1 illustrates a composition for supporting, optimizing and/or improving NAD+ concentrations in animals, mammals and/or humans, according to some example embodiments;

FIG. 2 illustrates a flowchart of at least some biological pathways associated with the NAD+ metabolome, according to some example embodiments;

FIG. 3 illustrates a nutritional label illustrating amounts of ingredients of a composition for supporting, optimizing and/or improving NAD+ concentrations in animals, mammals and/or humans, according to some example embodiments;

FIG. 4 illustrates a summary of a clinical trial of the efficacy of a composition for supporting, optimizing and/or improving NAD+ concentrations in animals, mammals and/or humans, according to some example embodiments;

FIG. 5 illustrates a summary of subjective effects on physical and mental energy, workout recovery and sleep quality, and enhanced gut health after several months of using a composition for supporting, optimizing and/or improving NAD+ concentrations, according to some example embodiments;

FIG. 6 illustrates a flowchart of a method of use for a composition for supporting, optimizing and/or improving NAD+ concentrations in animals, mammals and/or humans, according to some example embodiments; and

FIG. 7 illustrates a flowchart of a method of manufacture of a composition for supporting, optimizing and/or improving NAD+ concentrations in animals, mammals and/or humans, according to some example embodiments.

### DETAILED DESCRIPTION

Redundancy, also known by the science principle called degeneracy, is a characteristic feature of complex organic biological systems. We see this redundancy in our cells, which often have more than one way to do important tasks or make critical molecules. NAD+ is one of these critical molecules, so important to cellular function, your cells have more than one way to make it.

As will be described in more detail below, one or more compositions 100 comprising a synergistically effective amount of several different types of B-vitamins, minerals and/or bioactive compounds have been discovered to provide synergistic effects and unexpected results with respect to the support, optimization and/or improvement of NAD+ concentrations in animals, mammals and/or humans.

Specifically, in some embodiments as will be described in more detail below, compositions 100 comprise a synergistically effective amount of several different versions of vitamin B3, for example: nicotinamide riboside, nicotinamide and nicotinic acid to provide synergistic effects and unexpected results with respect to the support, optimization and/or improvement of NAD+ concentrations in animals, mammals and/or humans.

In some embodiments as will be described in more detail below, compositions 100 comprise a synergistically effective amount of the several versions of vitamin B3 and several other B-vitamins, for example, any one or more of: thiamin (vitamin B1), riboflavin (vitamin B2), pantothenic acid (vitamin B5), pyridoxine (vitamin B6), biotin (vitamin B7), folate (vitamin B9) and/or cobalamin (vitamin B12) to provide synergistic effects and unexpected results with respect to the support, optimization and/or improvement of NAD+ concentrations in animals, mammals and/or humans.

And, in yet other embodiments as will be described in more detail below, compositions 100 comprise a synergistically effective amount of the three versions of vitamin B3, the several other B-vitamins, at least one of magnesium, trans-resveratrol and caffeine to provide synergistic effects and unexpected results with respect to the support, optimization and/or improvement of NAD+ concentrations in animals, mammals and/or humans.

### Combining Several Forms of Vitamin B3 as Precursors to NAD+

Alone or in combination with one or more other B vitamins and/or at least one of magnesium, trans-resveratrol and caffeine, the present disclosure contemplates a synergistically effective amount of several versions of vitamin B3, for example: nicotinamide riboside, nicotinamide and nicotinic acid to provide synergistic effects and unexpected results with respect to the support, optimization and/or improvement of NAD+ concentrations in animals, mammals and/or humans.

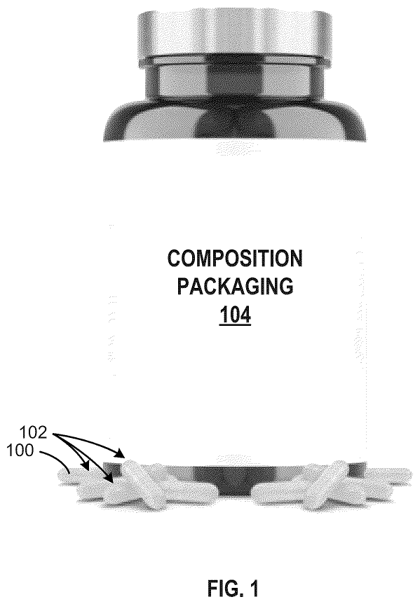

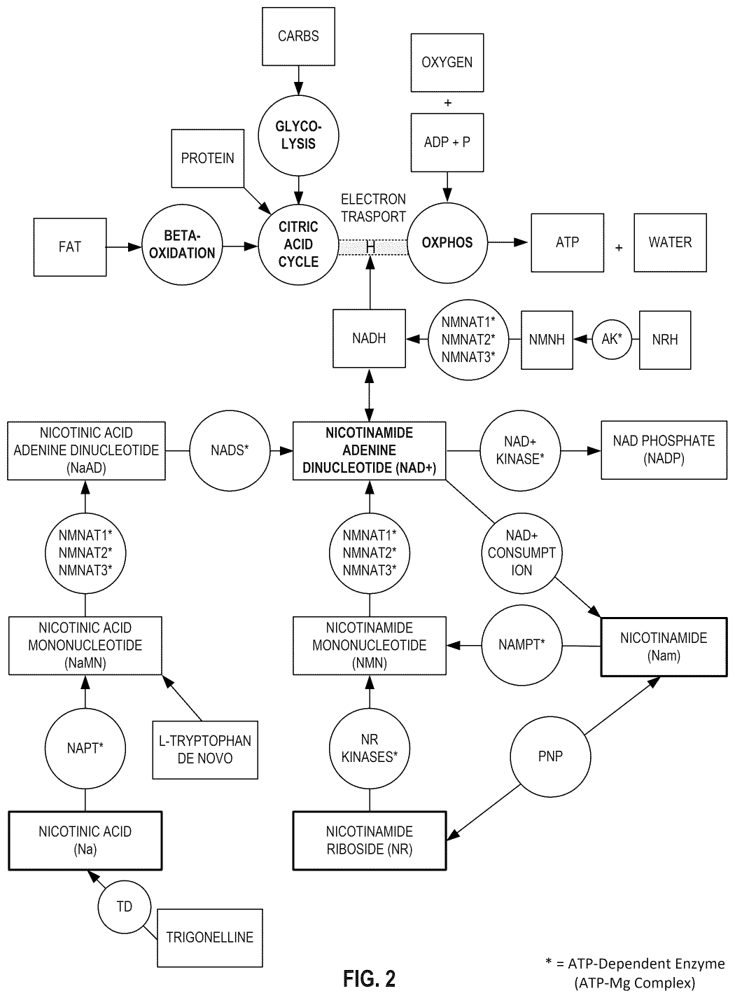

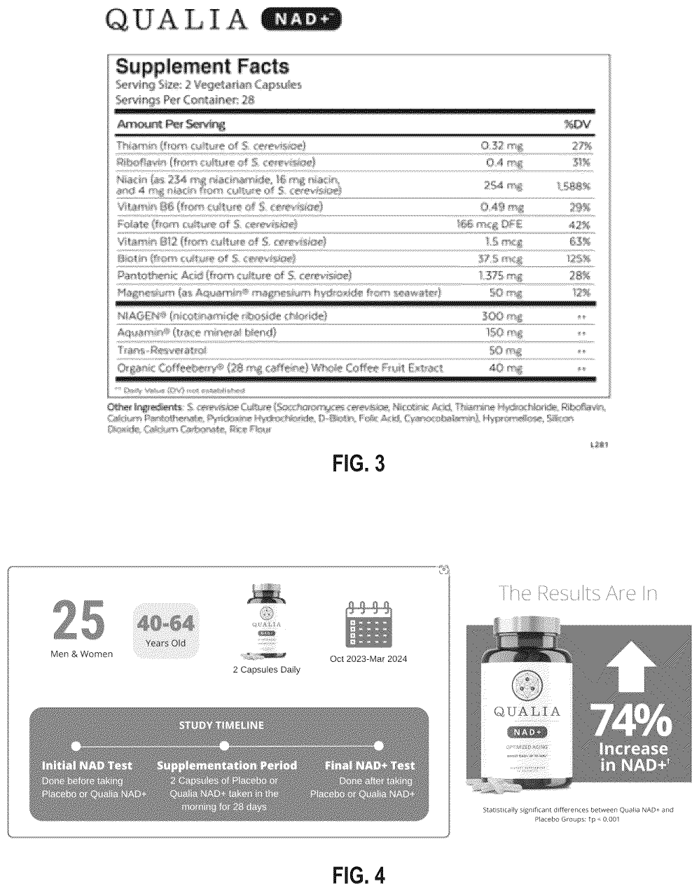

Turning to the flowchart of biochemical pathways associated with the NAD+ metabolome shown in FIG. 2, all forms of vitamin B3 can be used to make the larger NAD+ molecule. Accordingly, cells can make NAD+ from each of these three vitamin B3 precursors. This has prompted prior solutions to use single types of vitamin B3, as several precursors would be considered duplicative and, therefore, unnecessary and/or wasteful. However, cells make NAD+ from each of these three precursors through different biochemical pathways. And cells in some tissues rely more heavily on one pathway, while cells in other tissues may rely more heavily on another. Combining all three in a composition 100, see, e.g., FIG. 1 and/or the ingredient/nutrition label of FIG. 3, not only ensures a supply of each so that cells' NAD+ production, e.g., intracellular production and/or intracellular supply, is not limited by undersupply of any of these three precursors, it also, contrary to prior thinking and expectation, led to a synergistic increase in NAD+ that was larger than expected from prior scientific research, an unexpectedly large result. Also, supporting this multi-path redundancy cells rely on to make NAD+, by supplying the disclosed different types of vitamin B3, necessarily required action contrary to prior expectation that several precursors would be duplicative and, therefore, unnecessary and/or wasteful.

One form of vitamin B3 that can be converted to NAD+ is nicotinamide mononucleotide (NMN). However, the FDA's current position is that NMN cannot be lawfully sold as a dietary supplement.

Another form of vitamin B3 is nicotinamide riboside (NR), which can be directly converted into NMN by enzymes called nicotinamide riboside kinases (NRKs). And NMN is then converted into NAD+. Since our cells have these enzymes, nicotinamide riboside chloride has been studied as an NMN precursor. In fact, 300 mg of nicotinamide riboside chloride has been shown to increase NAD+ levels by an average of 48% in healthy and overweight adults over an 8-week period.

Prior to the discovery of the NRKs, it was widely believed that NR was cleaved to Nam and, so, NR was not considered worthwhile as a precursor supplement, as it would merely be converted to Nam. And, so, Nam could have just been provided. Accordingly, the established way to support NAD+ levels was to supplement the diet with nicotinic acid (Na, also called Niacin or, sometimes, flushing B3) or with nicotinamide (Nam, also called niacinamide or, sometimes, non-flushing B3). Each of these forms of vitamin B3 is converted to NAD+ through a different enzyme sequence that does not use the NRK enzymes.

Nicotinic acid (Na) is converted into NAD+ in a three-step process known as the Preiss-Handler pathway (nicotinic acid phosphor-ribosyl-transferase (NAPT or NAPRT) converts nicotinic acid to nicotinic acid mononucleotide (NaMN); NMN adenylyl transferases (NMNATs, e.g., NMNAT1, NMNAT2, NMNAT3) convert NaMN to nicotinic acid adenine dinucleotide (NaAD); and NAD+ synthase (NADS or NADSYN) converts NaAD to NAD+). Nicotinamide (Nam) is made into NAD+ in a two-step process (nicotinamide phosphor-ribosyl-transferase (NAMPT) converts Nam to nicotinamide mononucleotide (NMN); and NMNAT1-3 convert NMN to NAD+). This second step is shared with the nicotinamide riboside (NR) pathway. The pathways from NR to NMN to NAD+ and from Nam to NMN to NAD+ are called the Salvage Pathway.

NR can also be cleaved to form nicotinamide (Nam) by purine nucleoside phosphorylase (PNP), with Nam then being converted to NMN via NAMPT. There is also some recent evidence that inhibiting PNP may cause more NR to be converted to NMN by the NRKs. Accordingly, inhibitors of PNP may be useful to further boost NAD+ in the context of this disclosure.

Recently, the hydrogenated form of NR (NRH) and of NMN (NMNH) have also been found to increase NAD+ levels in a manner independent of NRKs or Nam salvage. Adenosine kinase (AK), not the NRKs, converts NRH to NMNH. NMNH is adenylated to NADH by NMN adenylyl transferases (NMNATs, e.g., NMNAT1, NMNAT2, NMNAT3) followed by oxidation to NAD+. This has been described as the reduced salvage pathway.

An intermediate in the Preiss-Handler pathway, nicotinic acid mononucleotide (NaMN), can be synthesized from L-tryptophan de novo through multiple enzymatic steps in the kynurenine pathway (KP). The NaMN produced can be used to produce NAD+ by proceeding through the final two enzyme steps of the Preiss-Handler pathway. L-tryptophan is not thought to contribute significantly to the overall NAD+ pool under normal circumstances and is a relatively inefficient way to make the NAD+ molecule, with about 60 mg of L-tryptophan needed to make the NAD+ equivalent to 1 mg of niacin in men.

Trigonelline (N-methyl nicotinic acid), a naturally occurring compound found in foods including fenugreek and coffee beans and also a metabolite produced by the gut microbiome and endogenous metabolism in humans, can be demethylated in the liver, by trigonelline demethylase (TD in FIG. 2), to produce nicotinic acid (Na), which can be used to produce NAD+.

In addition to the support for biological redundancy (i.e., degeneracy) and for the multiple pathways that supplying several different NAD+ precursors affords, nicotinamide riboside (NR), Niacinamide (Nam), and Nicotinic Acid (Na) have different pharmacokinetics after an oral dose, and have varied timing for how quickly they raise liver NAD+, when the peak of NAD+ occurs, and how long it remains elevated. See, e.g., Trammell S A, Schmidt M S, Weidemann B J, Redpath P, Jaksch F, Dellinger R W, Li Z, Abel E D, Migaud M E, Brenner C. Nicotinamide riboside is uniquely and orally bioavailable in mice and humans. Nat Commun. 2016 Oct. 10; 7:12948. doi: 10.1038/ncomms12948. PMID: 27721479; PMCID: PMC5062546. Specifically, this study observed: that NA administration produced the least increase in hepatic NAD+ in mice, and that production was 4-6 hours faster than NR and Nam in kinetics of hepatic NAD+ accumulation; a clear increase in hepatic NAD+2 hours post-Nam gavage, this gavage driving increased hepatic NAD+ accumulation from 2-8 hours, and peaking at 8 hours (and a ˜50% advantage of Nam over NA); and that NRE elevated hepatic NAD+ by more than fourfold and peaked 6 hours post-gavage. Accordingly, the inventors have found that skillfully combining several of these NAD+ precursors in the appropriate amounts complement each other's pharmacokinetics in an unexpectedly amplifying way that integrates the quick increase in liver NAD+ produced by Na with the delayed increase in liver NAD+ produced by NR resulting in sustaining an increase in higher NAD+ levels for longer periods of time than expected.

According to NIH fact sheets, 30-50 mg or more nicotinic acid typically causes flushing, in which the skin on the patient's face, arms, and chest turns a reddish color because of vasodilation of small subcutaneous blood vessels. This flushing can be accompanied by unpleasant burning, tingling, and itching sensations.

In order to provide a composition 100 that supports, optimizes and/or increases NAD+ concentrations in animals, mammals and/or humans, while simultaneously preventing and/or avoiding this flushing and the attendant unpleasant side effects, in some embodiments, a 25 mg serving of nicotinic acid, which is below the lowest amount reported by the NIH to typically cause flushing, is utilized in composition 100 along with a synergistically effective amount of added niacinamide and nicotinamide riboside sufficient to provide the desired support for, optimization of, and/or increase in NAD+ concentrations in animals, mammals and/or humans.

In some embodiments, an even smaller dose of nicotinic acid, for example 16 mgs, is utilized in composition 100 along with a synergistically effective amount of added niacinamide and nicotinamide riboside sufficient to provide the desired support for, optimization of and/or increase in NAD+ concentrations in animals, mammals and/or humans. As the NIH states that 30-50 mg of nicotinic acid typically causes flushing, and it is otherwise known that the more common symptom of flushing can occur at doses as low as 30 mg of nicotinic acid per day, the 25 mg dose of nicotinic acid would be expected to be small enough to prevent flushing. Accordingly, a dose of 16 mg of nicotinic acid would have previously been considered inappropriately, or unnecessarily, small or at the very least, moving in the wrong direction where the goal is to provide the desired support for, optimization of and/or increase in NAD+ concentrations in animals, mammals and/or humans. Accordingly, this disclosure describes compositions 100 comprising less than 30 mg of nicotinic acid, in some embodiments, between 8-29 mg of nicotinic acid, in some embodiments approximately 25 mg, and in some embodiments, approximately 16 mg-along with a synergistically effective amount of added niacinamide and nicotinamide riboside sufficient to provide the desired support for, optimization of and/or increase in NAD+ concentrations in animals, mammals and/or humans. For example, in some such embodiments, composition 100 also comprises between 100-500 mg of niacinamide, in some such embodiments, approximately 234 mg of niacinamide. And, in further example, in some such embodiments, composition 100 also comprises an amount of nicotinamide riboside chloride between 150-1000 mg (e.g., thereby supplying approximately 132-880 mg of nicotinamide riboside-based on testing of 4 of 5 samples of NR chloride showing 12% chloride content by weight and the other sample showing an 11% chloride content by weight, and based on NR chloride having a molecular weight of 290.7 g/mol, chloride having a molecular weight of 35.45 g/mol and, so, NR having a molecular weight of 255.25 g/mol, see, e.g., https://efsa.onlinelibrary.wiley.com/doi/epdf/10.2903/j.efsa.2019.5775). In some such embodiments, composition 100 also, or alternatively, comprises an amount of another nicotinamide riboside compound (e.g., nicotinamide riboside hydrogen malate) having a molar equivalent of nicotinamide riboside as is in between 150 mg and 1000 mg of nicotinamide riboside chloride. In some such embodiments, composition 100 comprises approximately 300 mg of nicotinamide riboside chloride or an amount of another nicotinamide riboside compound (e.g., nicotinamide riboside hydrogen malate) having an equivalent amount of nicotinamide riboside (e.g., moles) as in approximately 300 mg of nicotinamide riboside chloride

### Synergistic Support for Precursor Conversion to NAD+

Converting the raw materials of nicotinamide riboside, nicotinamide and nicotinic acid into the finished product NAD+ requires cellular work. And specific enzymes help this cellular work by catalyzing reactions that allow each precursor molecule to be made into a different molecule along the chemical pathway that ultimately converts each precursor into NAD+. Several such enzymes are shown in FIG. 2 for easy reference.

Accordingly, the present disclosure also contemplates that composition 100 comprises one or more of caffeine (e.g., whole coffee fruit extract, for example, from Coffeeberry®), trans-resveratrol, magnesium, and one or more of the entire family of B-vitamins to synergistically support the conversion of the nicotinamide riboside compound(s) (e.g., NR chloride and/or NR hydrogen malate), nicotinic acid (Na), and niacinamide (Nam) into NAD+. For example, in some embodiments, composition 100 may comprise several ingredients (e.g., one or more of the 11 described herein) that support the conversion of any of these three Vitamin B3 NAD+ precursors: magnesium, caffeine, trans-resveratrol, B1, B2, B4, B5, B6, B7, B9 and/or B12.

Further, cells don't just make NAD+ from these precursors as a dead-end pathway. Since NAD+ has many cellular jobs, NAD+ is often incorporated into different NAD-containing molecules, or consumed, leaving parts of it as leftovers, which must be recycled back to NAD+ to optimize NAD+ levels. In other words, even providing multiple precursors to NAD+ and one or more compounds that support their conversion to NAD+ may not be sufficient to wholistically supporting and/or boosting NAD+ concentrations in animals, mammals and humans and, in some cases, thereby maintain healthier states and reduce vulnerability to stressors of all types.

Accordingly, the present disclosure further contemplates composition 100 comprising additional components, compounds and/or molecules that support both the conversion of NAD+ into other molecules, and the recycling of any NAD+ “leftovers” back into NAD+, as described herein.

### Caffeine

Caffeine is one of the more widely used and studied cognitive (i.e., nootropic) and exercise (i.e., ergogenic) dietary supplement ingredients. Neuroscientists often group specific cognitive tasks into larger categories, one of which is often called “complex attention.” This includes much of what a person means when they say they'd like more focus. It's our ability to direct our cognitive resources where we want, for as long we want, while blocking out distractions. It also includes the capacity to respond quickly (i.e., reaction times and processing speed). Caffeine excels in promoting alertness and for tasks in the complex attention category. However, caffeine also has an unrelated benefit of increasing the ability of a specific enzyme to convert each of nicotinamide riboside, niacinamide and niacin into NAD+.

As shown in FIG. 2, nicotinamide riboside (NR) goes through a two-step conversion to NAD+, niacinamide (Nam) goes through a two-step conversion to NAD+, and niacin, or nicotinic acid (Na) requires a three-step conversion to NAD+. And each of these steps requires one or more enzymes to accelerate the conversion processes. While all three pathways start out using different enzymes, all three eventually converge on the same enzyme family called nicotinamide mononucleotide adenylyl transferases (NMNATs). Humans have three NMNAT variants (NMNAT1, NMNAT2, and NMNAT3), all of which catalyze the production of NAD+. NMNAT1 is expressed widely in the body. NMNAT2 is predominantly expressed in the brain. And NMNAT3 is widely expressed but has highest concentrations in liver, heart, skeletal muscles, and red blood cells. This is summarized in TABLE 1 below.

TABLE 1
NMNAT Subtypes, Tissue Activity, and Nutritional Support
NMNAT PROTEINS DO PART OF THE WORK THAT TURNS
NR, NIACIN, & NIACINAMIDE INTO NAD+

| What are the | Where Are They | |
|---|---|---|
| NMNAT Subtypes? | Mostly Expressed? | What Activates It? |
| NMNAT1 | Most Tissues | Magnesium (as ATP-Mg |
| | | complex) |
| NMNAT2 | Brain | Caffeine (found in |
| | | Coffeeberry ®); |
| | | Magnesium (as ATP-Mg |
| | | complex) |
| NMNAT3 | Heart, Liver, Red Blood | Magnesium (as ATP-Mg |
| | Cells, Skeletal Muscles | complex) |

A combination of nicotinamide riboside and caffeine has an additive effect on restoring NAD+ concentrations in skin and brain cells (both astrocytes and neural progenitor cells). Specifically, caffeine positively modulates the activity of the enzyme NMNAT2, an essential enzyme for NAD+ production in brain cells. In other words, caffeine supports cellular conversion of nicotinamide riboside, niacin and niacinamide into NAD+. Using an amount of whole coffee fruit extract (e.g., from Organic Coffeeberry®) having a standardized amount of caffeine also allows for the inclusion of coffee polyphenols, which support both mitochondrial performance and gut health. Accordingly, composition 100 for supporting, optimizing and/or increasing NAD+ concentrations in animals, mammals and/or humans may further comprise, in some embodiments, less than 50 mg of caffeine, in some embodiments, less than 40 mg of caffeine and, in some embodiments, approximately 28 mg of caffeine. These amounts of caffeine are all below what is considered the “nootropic” range of caffeine (50-200 mg), but still within the range where caffeine supports cognitive performance and mood. As stated above, in some embodiments, composition 100 comprises an amount of whole coffee fruit extract that includes the above-described ranges and/or amounts of caffeine.

### Resveratrol

Plants make trans-Resveratrol (trans-3, 5, 4′-trihydroxystilbene, usually called resveratrol) as part of their defense to pests, injuries, and environmental stressors like intense sunlight or drought. Plants make resveratrol to protect themselves and become more resilient, allowing them to survive in, and adapt to, less favorable conditions. In animals and humans, resveratrol supports a similar type of generalized resistance to many types of stress, appearing to act like a cellular and mitochondrial adaptogen.

One reason resveratrol may confer this resistance to stress is related to the idea of hormesis. Hormesis is a characteristic of many biological processes, stemming from the observation that some things we are exposed to (e.g., exercise, oxygen, and sunlight) don't follow a “more is better” rule. If we get too little exposure, we may not thrive. If we get too much, it may be toxic or cause a worsening of performance. A “just right amount,” within a range, or “hormetic zone,” is the sweet spot where best results occur. Many of the things we tend to think of as being stressful are actually hormetic, stimulating helpful adaptations and better overall fitness when exposure is in low-to-moderate amounts. Resveratrol has been proposed as just such a hormetic, providing a mild cellular and mitochondrial stress that causes cells to adapt.

Another reason resveratrol may confer this resistance to stress, one that overlaps with, but is somewhat different from, hormesis, is called xenohormesis. Hormesis is incorporated within xenohormesis, but so is the idea that animals co-evolved with plants, and that certain plant metabolites may be sending “early warning” signals to animals. Plants like grapes, berries and peanuts make more resveratrol when their environment is stressful, impoverished, or deteriorating. When animals eat these plants, they may also be consuming information about the environment the plant was grown in. The increased resveratrol acts as a message to the animal, an advance warning, that the environment the plant was grown in wasn't the most favorable, and to be prepared for your environment to become similarly less favorable. The animal's response to this advanced warning is a wide range of cellular and mitochondrial adaptations that result in being better prepared for adversity.

While hormesis and xenohormesis offer slightly different explanations, they both indicate a low-to-moderate amount of resveratrol. Accordingly, composition 100 for supporting, optimizing and/or increasing NAD+ concentrations in animals, mammals and/or humans may further comprise, in some embodiments, between 8-200 mg of resveratrol and, in some embodiments, approximately 50 mg of resveratrol. Not only does resveratrol support cellular and mitochondrial adaptations to stress, acting somewhat akin to a mild calorie restriction mimetic in terms of the many pathways and behaviors it modifies, it also supports an enzyme called NAMPT, which is the first step in the NAD+ salvage pathway that converts niacinamide (Nam) into NAD+ and is central to re-making NAD+. In some embodiments, composition 100 for supporting, optimizing and/or increasing NAD+ concentrations in animals, mammals and/or humans may include a high-purity resveratrol made using an innovating natural yeast fermentation process.

### Magnesium

Magnesium is one of the most abundant minerals in the body and is vital for the functioning of all living cells, as it is used in more than 300 enzymes and it is needed to make some neurotransmitters in the brain and gut. About ⅓ of intracellular magnesium is held inside mitochondria. Accordingly, magnesium is essential for mitochondrial performance. Magnesium also plays a large role in breaking down sugars (e.g., the ATP-producing process of glycolysis) and, so, is essential for energy metabolism. In fact, magnesium is bound to ATP in a complex that is required to speed up the work of hundreds of enzymes. The recommended dietary allowances for magnesium in adults vary from 310-420 mg, depending upon age and gender. However, a majority of Americans (of all ages) fall short of this amount of magnesium from the foods they eat.

We need NAD+ to make ATP. However, what does not get mentioned is that this ATP-magnesium complex is also required for much of the cellular work needed to make NAD+ from each of the three Vitamin B3 NAD+ precursors. As previously stated, the conversion pathway of nicotinamide riboside into NAD+ comprises two enzymatic steps, and both require the ATP-magnesium complex. The conversion pathway of niacinamide (Nam) to NAD+ also comprises two enzymatic steps, and both are dependent on the ATP-magnesium complex. The conversion pathway of nicotinic acid into NAD+ comprises three enzymatic steps, and all three of them use the ATP-magnesium complex to catalyze the reactions. Further, converting NAD+ into the nicotinamide adenine dinucleotide phosphate (NADP+), a form that supports cellular protection and detoxification mechanisms, also requires ATP.

Scientists sometimes use “NAD+ metabolome” to refer to NAD+, all of the molecules used to make it, as well as the various forms the NAD+ molecule converts into. See, e.g., FIG. 2. A 2018 scientific study reported that, not only did plasma NAD+ decline with age, several other molecules in the NAD+ metabolome declined with age, e.g., nicotinic acid adenine dinucleotide (NaAD) and NADP+. See, e.g., Clement J, Wong M, Poljak A, Sachdev P, Braidy N. The Plasma NAD+ Metabolome Is Dysregulated in “Normal” Aging. Rejuvenation Res. 2019 April; 22 (2): 121-130. doi: 10.1089/rej.2018.2077. Epub 2018 Oct. 23. PMID: 30124109; PMCID: PMC6482912. However, other molecules within the metabolome increased, rather than decreased, with age, e.g., nicotinamide mononucleotide (NMN) and nicotinic acid mononucleotide (NaMN). Why were the amounts of some molecules in the plasma NAD+ metabolome too high but others too low? Specifically, if there was more than enough of one molecule, and not enough of the next molecule in the conversion pathway, why was this concentration gradient not driving conversion of the former into the latter? And what was allowing the former molecule to continue to build up despite its increasing concentration? In trying to make sense of these findings, the inventor of this invention researched the enzymes that create flux through the NAD+ metabolome and what factors might impact flux in the plasma (i.e., extracellularly) versus intracellularly. In scientific articles, it's common to see drawings of the NAD+ metabolome that name the enzymes and molecules. It is uncommon to find drawings that provide more detail on what may impact the function of these enzymes or act as activators, including the role of potential cofactors such as magnesium.

The inventor of the invention(s) described by the instant disclosure has discovered that ATP energetics impacts the flux through the NAD+ metabolome. The role of ATP has been almost entirely ignored in scientific research related to the NAD+ metabolome. Most scientific studies that show diagrams of the NAD+ metabolome don't include which enzymes are ATP-dependent in the diagrams or mention their role in the text of the publication. The inventor identified three scientific articles that did place ATP in the diagrams of the NAD+ metabolome; however, they were incomplete and disagreed with each other for several enzymes (NAMPT and NADS being two examples). With further investigation, the inventor has concluded that all intracellular enzymes that produce NAD+, whether from nicotinic acid (NA), niacinamide (Nam), or nicotinamide riboside (NR), depend on ATP as part of the enzymatic reaction. Synthesis of one mole of NAD+ from NA requires three moles of ATP; synthesis of one mole of NAD+ from Nam requires two moles of ATP; and synthesis of one mole of NAD+ from NR requires two moles of ATP. The reduced salvage pathway, starting from nicotinamide riboside hydrogen (NRH), is also reliant on APT. This conclusion of the central role of ATP in the enzymes involved in NAD+ synthesis has not previously been made in the scientific literature to the inventor's knowledge, nor has the conclusion that ATP would differently impact the NAD+ metabolome inside and outside cells.

But flux through the NAD+ metabolome enzymes intracellularly and extracellularly is not necessarily the same, because ATP is made intracellularly, with most made within the mitochondria. ATP is not made outside cells and is thought to be released to the extracellular space from dying or damaged cells. The intracellular versus extracellular availability of ATP would be expected to produce differential build-ups of molecules in the NAD+ metabolome inside cells versus in plasma. The inventors have not found any scientific articles that have concluded that all enzymes involved in creating NAD+ from NA, Nam, NR, and NRH rely on ATP. In fact, the scientific reviews that have drawn ATP in the NAD+ metabolome have failed to correctly identify the totality of the role ATP has in the NAD+ metabolome. See, e.g., Giner M P, Christen S, Bartova S, Makarov M V, Migaud M E, Canto C, Moco S. A Method to Monitor the NAD+ Metabolome—From Mechanistic to Clinical Applications. Int J Mol Sci. 2021 Sep. 30; 22 (19): 10598. doi: 10.3390/ijms221910598. PMID: 34638936; PMCID: PMC8508997; Nikiforov A, Kulikova V, Ziegler M. The human NAD metabolome: Functions, metabolism and compartmentalization. Crit Rev Biochem Mol Biol. 2015; 50 (4): 284-97. doi: 10.3109/10409238.2015.1028612. Epub 2015 Apr. 2. PMID: 25837229; PMCID: PMC4673589; and Chiarugi A, Dolle C, Felici R, Ziegler M. The NAD metabolome—a key determinant of cancer cell biology. Nat Rev Cancer. 2012 November; 12 (11): 741-52. doi: 10.1038/nrc3340. Epub 2012 Sep. 28. PMID: 23018234. A 1977 study concluded ATP levels are closely correlated with NAD and NADP levels in red cells. See, e.g., Pescarmona G P, Bracone A, David O, Sartori M L, Bosia A. Regulation of NAD and NADP synthesis in human red cell. Acta Biol Med Ger. 1977; 36 (5-6): 759-63. PMID: 23635. And a 2013 scientific publication identified mitochondrial dysfunction, which includes reduced ATP, as a characteristic of aging. See, e.g., López-Otín C, Blasco M A, Partridge L, Serrano M, Kroemer G. The hallmarks of aging. Cell. 2013 Jun. 6; 153 (6): 1194-217. doi: 10.1016/j.cell.2013.05.039. PMID: 23746838; PMCID: PMC3836174. Accordingly, using an “ATP lens” the emerging picture becomes one of ATP having a central role in flux through the NAD+ metabolome with insufficient ATP activity to drive the enzymes that make the NAD+ metabolome function well as we get older, potentially independent of the supply of any of the three B3 vitamin precursors to NAD+.

Accordingly, making NAD+, and obtaining a youthful flux through the NAD+ metabolome, is utterly reliant on ATP, and ATP is reliant on magnesium for its activity. Specifically, the ATP-magnesium complex is needed for conversion of: (1) nicotinic acid (Na) to NAD+, (2) nicotinamide riboside (NR) to NMN, and (3) NMN, whether made from niacinamide (Na) or from nicotinamide riboside (NR), to NAD+. In FIG. 2, several of the enzymes involved in the NAD+ metabolome that are ATP-dependent (e.g., ATP-Mg complex dependent) are illustrated with a “*” Magnesium is also an essential cofactor in five of the eleven enzyme steps required for glycolysis, the first of several linked metabolic pathways that convert glucose into ATP. There is a net gain of two molecules of ATP produced in glycolysis from one molecule of glucose, and glycolysis sets the stage to gain an additional 28-36 ATP molecules from one glucose in subsequent linked energy metabolism pathways. Accordingly, composition 100 for supporting, optimizing and/or increasing NAD+ concentrations in animals, mammals and/or humans may further comprise, in some embodiments, between 25-167 mg of magnesium and, in some embodiments, approximately 50 mg of magnesium to, for example, bridge the gap between RDA and actual magnesium intake from food sources. In some embodiments, composition 100 comprises an amount of Aquamin® Mg having the above-described range and/or amount(s) of magnesium in the bioavailable form of magnesium hydroxide along with lesser amounts of 71 other minerals, many of which play roles in cellular and mitochondrial function. For example, the magnesium in Aquamin® Mg is captured from clean sea water off the Irish coast.

### Fermented B Complex

A generalizable principle in biology is that nothing is more central to life and health than energy. Accordingly, energy metabolism governs all biochemistry! Adenosine Triphosphate (ATP) is the energy currency of cells because cells can make and break ATP extremely quickly. In fact, a working muscle cell makes and uses about 10 million molecules of ATP every second! And each ATP molecule is recycled some 1000 to 1500 times per day. This means the human body turns over its entire weight in ATP every day.

As stated above, ATP is indispensable for making NAD+ and for healthy flux of molecules through the NAD+ metabolome but NAD+ is also required to make ATP. So, how is ATP generated? The bound form of energy in both plants and animals can be simplified to carbon and hydrogen, and in some ways, it is this hydrogen (and the energy stored in its bonds with other molecules including carbon) that ultimately fuels our body. Carbohydrates are just hydrated chains or rings of carbon, and fats are essentially strings of carbon with varying degrees of “saturation” of bonded hydrogen atoms (related to the number of carbon-carbon double bonds in the fatty acid chain). During metabolism, NAD+ accepts these hydrogens, becoming NADH, and NADH carries the hydrogen (and electrons) to the outer membrane of the mitochondria where they are used to fuel the making of ATP.

These mitochondria, and four mitochondria-linked energy metabolism pathways that all ultimately rely on NAD+, are central to the function and utility of ATP. As shown in FIG. 2, one of these linked pathways is “glycolysis,” the process that splits carbohydrates, with the metabolites (pyruvate) being shuttled into the mitochondria for intake into one of the other pathways below. Two net molecules of ATP are gained in glycolysis from one molecule of glucose.

The other three mitochondria-linked pathways occur in the mitochondria. One is called “beta-oxidation,” where fatty acids are broken down two carbon “links” at a time to enter aerobic metabolism. Another, which is a part of aerobic metabolism, is called the “Krebs cycle” or “citric acid cycle,” where metabolites of carbohydrates, fat and protein are broken down into carbon dioxide and energetic hydrogens (and electrons). Each turn of the Krebs cycle can produce one GTP (or ATP). And the last of the four mitochondria-linked pathway occurs in the folds (called the cristae) of the inner membranes of the mitochondria and is called “oxidative phosphorylation” (OXPHOS) or “electron transport,” where these hydrogens and electrons, being carried by NAD (as NADH) and vitamin B2-dependent flavin adenine dinucleotide (FAD) (as FADH2), are finally used to fuel the production of the majority of ATP by snapping free phosphate back onto slightly-discharged adenosine diphosphate (ADP). For glucose, as an example, one glucose molecule proceeding through glycolysis, the citric acid cycle, and oxidative phosphorylation is estimated to produce a net gain of between 30 and 38 ATP. But only 2-4 of the ATP are produced in glycolysis and the citric acid (Krebs) cycle. It's the NADH and FADH2 molecules produced in glycolysis and the citric acid (Krebs) cycle that contribute most of the net gain of ATP when they go through OXPHOS. These four linked pathways are, essentially, what cells use to transform the stored energy in food into the ATP that fuels cellular work, including the production and maintenance of NAD+ and the various molecules comprising the pathways of the NAD+ metabolome.

B-vitamins are a family of eight vitamins, all of which are essential for energy metabolism and/or mitochondrial function. Specifically, Vitamins B1, B2, B3, and B5 support glycolysis (as does magnesium); Vitamin B6 is used to convert glycogen (a storage sugar in animals) into the glucose used in glycolysis; Vitamin B2, B3, B5, and biotin are required for beta-oxidation; Vitamins B1, B2, B3, B5, B12 and biotin (and once again magnesium) are needed for the Krebs cycle; and vitamins B2, B3, and biotin (as well as magnesium) support the final linked pathway, oxidative phosphorylation (OXPHOS). While folate (Vitamin B9) isn't directly used in energy production, it is essential for the health of the mitochondria, in which all of the four linked pathways except glycolysis are carried out.

In some embodiments, a B-vitamin enriched postbiotic yeast (e.g., Saccharomyces cerevisiae) may be utilized to deliver fermented, more bioavailable B vitamins in a food matrix composition 100 for supporting, optimizing and/or increasing NAD+ concentrations in animals, mammals and/or humans as described anywhere in this disclosure. For example, during the fermentation process, the yeast is supplemented with specific levels of B vitamins, allowing the B vitamins to interact with, and be incorporated into, complexes the yeast would use to make its own ATP. The gentle processing of the yeast preserves the minerals, β-glucans, peptides, and nucleotides that naturally occur in these nutritional yeasts. Supplying the B-vitamins in this form also provides the advantage of providing the B-vitamins, in these compositions, in the exact forms they would be found in the above-described metabolic pathways in our cells, which increases bioavailability. As an example, like humans, yeast requires NAD+. And, also like humans, yeast has multiple pathways for making NAD+. This means that the vitamin B3 activity is spread through the entire NAD+ metabolome. Building blocks, intermediate molecules, NAD+, and NAD+'s metabolites are present in this nutritional yeast. By including a B-vitamin enriched postbiotic yeast in compositions described anywhere herein to also deliver fermented forms of vitamin B3 (in addition to other B vitamins as described anywhere herein), we are supplying and/or supporting a 4th way to make NAD+, by supplying the NAD-containing molecules in the many forms that occur in living cells across the NAD+ metabolome.

For example, in some embodiments, composition 100 for supporting, optimizing and/or increasing NAD+ concentrations in animals, mammals and/or humans as described anywhere in this disclosure may include between 28-125% of the daily value, depending on the B vitamin, either as separate contributions of predetermined and/or desired amounts of one or more of the B-vitamins, or in the form of nutritional yeast (as described anywhere herein) comprising a predetermined or desired amount of any one or more of the B-vitamins.

For example, in some embodiments, composition 100 for supporting, optimizing and/or increasing NAD+ concentrations in animals, mammals and/or humans may comprise between 0.16-6.0 mg of thiamin (Vitamin B1), and in some embodiments, approximately 0.32 mg of thiamin (Vitamin B1).

In some embodiments, composition 100 for supporting, optimizing and/or increasing NAD+ concentrations in animals, mammals and/or humans may comprise between 0.2-6.0 mg of riboflavin (Vitamin B2), and in some embodiments, approximately 0.4 mg of riboflavin (Vitamin B2).

In some embodiments, composition 100 for supporting, optimizing and/or increasing NAD+ concentrations in animals, mammals and/or humans may comprise between 0.65-10.0 mg of pantothenic acid (Vitamin B5), and in some embodiments, approximately 1.375 mg of pantothenic acid (Vitamin B5).

In some embodiments, composition 100 for supporting, optimizing and/or increasing NAD+ concentrations in animals, mammals and/or humans may comprise between 0.24-8.0 mg of pyridoxine (Vitamin B6), and in some embodiments, approximately 0.49 mg of pyridoxine (Vitamin B6).

In some embodiments, composition 100 for supporting, optimizing and/or increasing NAD+ concentrations in animals, mammals and/or humans may comprise between 0.15-300 mcg of biotin (Vitamin B7), and in some embodiments, approximately 37.5 mcg of biotin (Vitamin B7).

In some embodiments, composition 100 for supporting, optimizing and/or increasing NAD+ concentrations in animals, mammals and/or humans may comprise between 50-800 mcg Dietary Folate Equivalents (DFE) of folate (Vitamin B9), and in some embodiments, approximately 166 mcg DFE of folate (Vitamin B9).

In some embodiments, composition 100 for supporting, optimizing and/or increasing NAD+ concentrations in animals, mammals and/or humans may comprise between 0.5-10 mcg of cobalamin (Vitamin B12), and in some embodiments, approximately 1.5 mcg of cobalamin (Vitamin B12).

In some embodiments, composition 100 comprises postbiotic yeast comprising the above ranges or approximately amounts of at least one of thiamin (vitamin B1), riboflavin (vitamin B2), nicotinamide riboside, nicotinamide and/or nicotinic acid (vitamin's B3), pantothenic acid (vitamin B5), pyridoxine (vitamin B6), biotin (vitamin B7), folate (vitamin B9) and cobalamin (vitamin B12).

In some embodiments, composition 100 is disposed within at least one pharmaceutically acceptable vehicle 102, for example, capsule form (e.g., oral capsule form) and, in some embodiments, further packaged in packaging 104, which is shown as a bottle in FIG. 1, but which may alternatively be any suitable packaging such as blister packs, pouches and/or individual servings packaged in sealed bags.

### Scientific Studies on Efficacy of Compositions as Described Herein

Compound 100 is configured to help boost NAD+ by up to 50%, though it has been shown to increase NAD+ by an average of 74% in one double-blinded, placebo-controlled study, and by as much as 156% after 20 days of supplementation by one of the inventors of compound 100. Compound 100 is also configured to help combat the aging process, help activate cellular longevity processes, support cellular energy production, and/or support mitochondrial function.

A double-blind, placebo-controlled clinical trial of Qualia NAD+ was carried out with healthy individuals aged 40 to 64 years old between October 2023 and March 2024. Qualia NAD+™ is a composition for supporting, optimizing and/or increasing NAD+ concentrations in animals, mammals and/or humans, which comprises the 3 above-described Vitamin B3 precursors of NAD+ and the other 11 above-described supporting components in the concentrations illustrated in the label shown in FIG. 3.

Preliminary results from 25 participants who have completed the study have shown a statistically significant increase in NAD+ levels compared to placebo (p<0.001). Specifically, NAD+ was increased by 74%, on average, after taking Qualia NAD+once daily for 4 weeks, for example, as summarized in FIG. 4.

There were two groups in the study: one group that took Qualia NAD+™ capsules and the other group that took placebo capsules. Participants were randomly assigned to one of the two groups. The study was double-blinded, meaning neither the researchers conducting the study nor the participants knew who received Qualia NAD+™ or placebo capsules until after the participants had completed the 28-day study protocol (October 2023-March 2024). And both groups were instructed to take two capsules every day, anytime in the morning, with or without food. The primary study endpoint was the change in NAD+ levels, which was determined by comparing initial and final NAD+ levels using at-home finger stick blood kits.

A group of 92 adults were recruited and sent at-home finger stick blood kits to measure initial blood NAD+ levels prior to beginning supplementation. Forty-nine participants successfully completed the initial test to obtain their baseline NAD+ measurements and were instructed to take Qualia NAD+ or placebo. Twenty-five of the 49 participants successfully completed a second NAD+ test after taking Qualia NAD+ or a placebo for 28 days. Data analysis is based on these 25 participants who had both initial and final NAD+ measurements.

The participants, who had both initial and final NAD+ measurements, ranged from 40 to 64 years old, with an average age of 49.5 years. Participants were a mix of females (N=14) and males (N=11). The average increase in whole blood NAD+ levels from baseline in the group of participants taking Qualia NAD+ was 74%. This increase was statistically significant (p <0.001) compared to the placebo group. These results are summarized in Table 2 below.

TABLE 2
Initial and Final NAD+ Levels in Qualia NAD+ & Placebo Groups

| | Qualia NAD+ Group | Placebo Group |
|---|---|---|
| Initial NAD+ Levels | 19.64 | 19.06 |
| (μmol/L) | | |
| Final NAD+ Levels | 34.20† | 19.77 |
| (μmol/L) | | |
| Percent Change | 74% Increase† | 4% Increase |

†p < 0.001 comparing NAD+ level increase between Qualia NAD+ and placebo

This NAD+ increase of 74% found in this study is significantly greater than what has been reported to occur with a similar amount of nicotinamide riboside given alone (in which NAD+ was increased by about 50%). See, Conze D, Brenner C, Kruger CL. Safety and Metabolism of Long-term Administration of NIAGEN (Nicotinamide Riboside Chloride) in a Randomized, Double-Blind, Placebo-controlled Clinical Trial of Healthy Overweight Adults. Sci Rep. 2019; 9:9772. doi: 10.1038/s41598-019-46120-z.

Further, another study, using a 250 mg dose of NR+50 mg of pterostilbene, increased NAD by approximately 40%. See, Dellinger, R. W., Santos, S. R., Morris, M., Evans, M., Alminana, D., Guarente, L., & Marcotulli, E. (2017). Repeat dose NRPT (nicotinamide riboside and pterostilbene) increases NAD+ levels in humans safely and sustainably: A randomized, double-blind, placebo-controlled study. Npj Aging and Mechanisms of Disease, 3 (1). https://doi.org/10.1038/s41514-017-0016-9.

By contrast, Qualia NAD+™ has been shown to increase NAD+ by ˜74%, a significantly greater percentage than prior formulations above. See, https://www.qualialife.com/studies/qualia-nad-placebo-controlled-clinical-study, hereby incorporated in its entirety). This indicates that the comprehensive approach of compound 100 combining nicotinamide riboside with two additional NAD+ precursors and other specific ingredients intended to support the making of NAD+, has unexpected, synergistic, and additive benefits for supporting, optimizing and/or increasing NAD+ concentrations in humans, compared to nicotinamide riboside given alone or with pterostilbene.

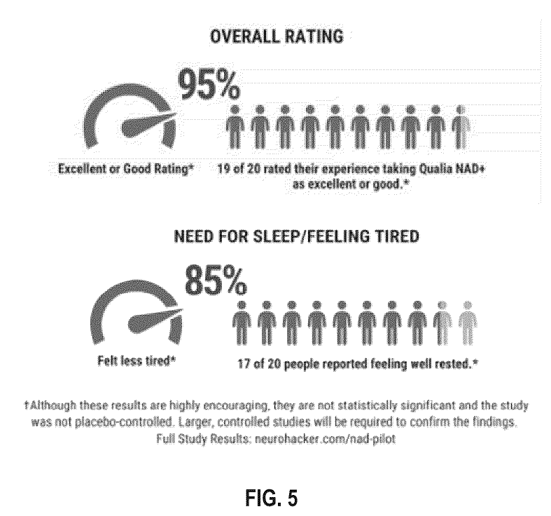

Additionally, many people have reported subjective effects after several months of using compound 100, in concentrations as shown on the label of FIG. 2, including: enhanced physical and mental energy, workout recovery and sleep quality, and enhanced gut health, for example as summarized in FIG. 5. Specifically, a Qualia NAD+ Pilot Study of 20 healthy adults (age range 40 to 72) were instructed to take Qualia NAD+ daily for 10 consecutive days. 91% of the men and 100% of the women improved their Healthy Aging Scores (which assesses the impact of aging on physical health, emotional well-being, and sexual function), 19 of the 20 participants rated their experience taking compound 100 as excellent or good, and 17 of the 20 reported feeling well rested and less tired. Although these results are highly encouraging and were statistically significant changes from baseline, the study was not placebo-controlled.

### Method of Use

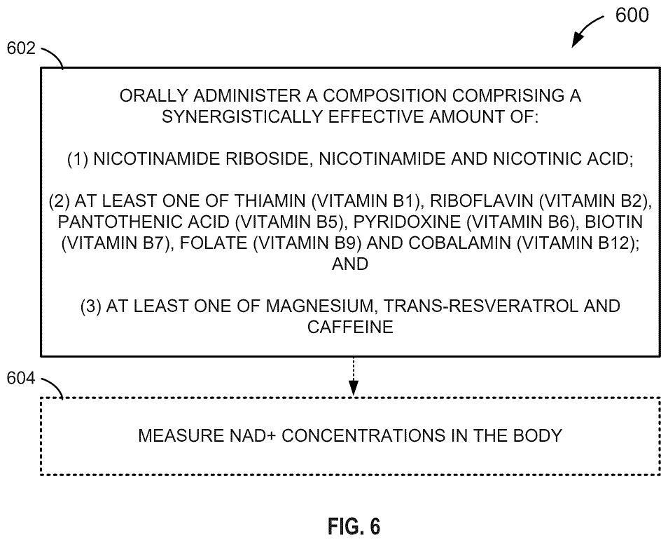

The disclosure now turns to FIG. 6, which illustrates a flowchart 600 related to a method of supporting nicotinamide adenine dinucleotide (NAD+) concentrations as described anywhere in this disclosure. Although particular steps are described herein, the present application is not so limited and alternative methods may include a subset of these steps, in the same or different order, and may additionally include one or more additional steps not described herein.

Step 602 includes orally administering a composition comprising a synergistically effective amount of: (1) nicotinamide riboside, nicotinamide and nicotinic acid; (2) at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and (3) at least one of magnesium, trans-resveratrol and caffeine.

As described anywhere in this disclosure, the composition, e.g., composition 100, may be in the form of a powder admixture of the included ingredients, although the present disclosure is not so limited and composition 100 may be in any other suitable form, or admixture of forms, such as liquid and/or solid pill or tablet.

In some embodiments, composition 100 is further disposed within at least one pharmaceutically acceptable vehicle, for example an oral capsule such as a vegetarian capsule. For example, a dose or serving of composition 100 may be disposed within one (or more) such capsules that may be swallowed by a user. Such dosing may occur at any time during the day, in some embodiments, preferably in the morning, with or without food, since circadian (i.e., body clock) research in animals suggests that NAD+ boosting compounds offer better support for metabolic health when taken in the morning. Taking compound 100 at the start of the day may make the best use of these natural rhythms. As stated previously herein, supplementing such a composition 100 daily for 28 days has been shown to increase NAD+ concentrations by an average of 74% in a double-blinded, placebo-controlled study.

In some embodiments, flowchart 600 may further include a step 604, which may include measuring NAD+ concentration in the body. For example, after a period of time for example 28 days of daily supplementing compound 100, NAD+ can easily be measured by at home testing kits such as Jinfiniti™, or through a physician. Monitoring changes to NAD+ concentrations before and after regular supplementation of compound 100 may provide direct feedback as to the effectiveness of compound 100 at increasing NAD+ concentrations.

### Method of Manufacture

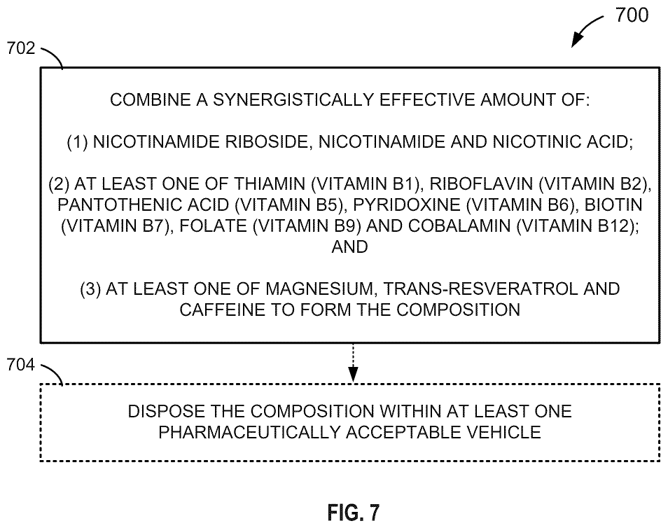

The disclosure now turns to FIG. 7, which illustrates a flowchart 700 related to a method of manufacturing a composition for supporting nicotinamide adenine dinucleotide (NAD+) concentrations as described anywhere in this disclosure. Although particular steps are described herein, the present application is not so limited and alternative methods may include a subset of these steps, in the same or different order, and may additionally include one or more additional steps not described herein.

Step 702 includes combining a synergistically effective amount of: (1) nicotinamide riboside, nicotinamide and nicotinic acid; (2) at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and (3) at least one of magnesium, trans-resveratrol and caffeine to form the composition.

As described anywhere in this disclosure, composition 100 may be in the form of a powder admixture of the included ingredients, although the present disclosure is not so limited and composition 100 may be in any other suitable form, or admixture of forms, such as liquid and/or solid pill or tablet.

In some embodiments, flowchart 700 includes step 704, disposing composition 100 within at least one pharmaceutically acceptable vehicle. In some embodiments, such a pharmaceutically acceptable vehicle comprises one or more oral capsules such as vegetarian capsules. For example, a dose or serving of composition 100 may be disposed within one (or more) such capsules that may be swallowed by a user.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to diagrams, operational descriptions, and method claims, the order in which the steps are presented herein shall not mandate that various embodiments be implemented to perform the recited functionality in the same order unless the context dictates otherwise.

Although the disclosure is described above in terms of various embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “typical,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “element” does not imply that the components or functionality described or claimed as part of the element are all configured in a common package. Indeed, any or all of the various components of an element can be combined in a single package or separately maintained and can further be distributed in multiple groupings or packages or across multiple locations.

Additionally, the various embodiments set forth herein are described in terms of exemplary diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A composition for supporting nicotinamide adenine dinucleotide (NAD+) concentrations in humans, the composition comprising a synergistically effective amount of:
nicotinamide riboside, nicotinamide and nicotinic acid;
at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and
at least one of magnesium, trans-resveratrol and caffeine.

2. The composition of claim 1, comprising:
less than 30 mg of nicotinic acid;
between 100-500 mg of niacinamide;
between 132-880 mg of nicotinamide riboside or between 150-1000 mg of nicotinamide riboside chloride;
between 0.16-6 mg of thiamin (vitamin B1);
between 0.2-6 mg of riboflavin (vitamin B2);
between 0.65-10 mg of pantothenic acid (vitamin B5);
between 0.24-8 mg of pyridoxine (vitamin B6);
between 0.15-30 mcg of biotin (vitamin B7);
between 50-800 mcg of folate (vitamin B9);
between 0.5-10 mcg of cobalamin (vitamin B12);
less than 50 mg of caffeine;
between 8-200 mg of trans-resveratrol; and
between 25-167 mg of magnesium.

3. The composition of claim 2, comprising:
between 8-29 mg of nicotinic acid;
approximately 234 mg of niacinamide; and
approximately 264 mg of nicotinamide riboside or approximately 300 mg of nicotinamide riboside chloride.

4. The composition of claim 2, comprising:
approximately 25 mg of nicotinic acid;
approximately 234 mg of niacinamide; and
approximately 264 mg of nicotinamide riboside or approximately 300 mg of nicotinamide riboside chloride.

5. The composition of claim 2, comprising:
approximately 16 mg of nicotinic acid;
approximately 234 mg of niacinamide; and
approximately 264 mg of nicotinamide riboside or approximately 300 mg of nicotinamide riboside chloride.

6. The composition of claim 2, comprising:
approximately 0.32 mg of thiamin (vitamin B1);
approximately 0.4 mg of riboflavin (vitamin B2);
approximately 1.375 mg of pantothenic acid (vitamin B5);
approximately 0.49 mg of pyridoxine (vitamin B6);
approximately 37.5 mcg of biotin (vitamin B7);
approximately 166 mcg DFE of folate (vitamin B9); and
approximately 1.5 mcg of cobalamin (vitamin B12).

7. The composition of claim 1, wherein the composition comprises postbiotic yeast comprising the at least one of riboflavin (vitamin B2), pantothenic acid (vitamin B5), pyridoxine (vitamin B6), biotin (vitamin B7), folate (vitamin B9) and cobalamin (vitamin B12).

8. The composition of claim 7, wherein the postbiotic yeast comprise Saccharomyces cerevisiae.

9. The composition of claim 2, comprising:
approximately 28 mg of caffeine;
approximately 50 mg of trans-resveratrol; and
approximately 50 mg of magnesium.

10. The composition of claim 2, comprising less than 40 mg of caffeine.

11. The composition of claim 10, comprising between 10-39 mg of caffeine.

12. composition of claim 2, wherein the composition comprises whole coffee fruit extract, which comprises the caffeine.

13. The composition of claim 1, further disposed within at least one pharmaceutically acceptable vehicle.

14. The composition of claim 1, wherein the composition is a powder.

15. A method of supporting nicotinamide adenine dinucleotide (NAD+) concentrations in humans, the method comprising:
orally administering a composition comprising a synergistically effective amount of:
nicotinamide riboside, nicotinamide and nicotinic acid;
at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and
at least one of magnesium, trans-resveratrol and caffeine.

16. method of claim 15, further comprising measuring NAD+ concentration in the body.

17. A method of manufacturing a composition for supporting nicotinamide adenine dinucleotide (NAD+) concentrations in humans, the method comprising:
combining a synergistically effective amount of:
nicotinamide riboside, nicotinamide and nicotinic acid;
at least one of thiamin (vitamin B1), riboflavin (vitamin B2), pantothenic acid (vitamin B5), pyridoxine (vitamin B6), biotin (vitamin B7), folate (vitamin B9) and cobalamin (vitamin B12); and
at least one of magnesium, trans-resveratrol and caffeine.

18. The method of claim 17, further comprising disposing the composition within at least one pharmaceutically acceptable vehicle.
